(19) United States
(12) Patent Application Publication
(10) Pub. No.: US 2016/0256066 A1
(43) Pub. Date: Sep. 8, 2016

(54) METHOD AND SYSTEM TO MEASURE PHYSIOLOGICAL SIGNALS OR TO ELECTRICALLY STIMULATE A BODY PART

(71) Applicant: CSEM CENTRE SUISSE D'ELECTRONIQUE ET DE MICROTECHNIQUE SA - RECHERCHE ET DEVELOPPMENT, Neuchatel (CH)

(72) Inventors: Olivier Chetelat, Cudrefin (CH); Damien Ferrario, Vevey (CH); Thomas Parkel, Jona (CH); David Hasler, Neuchatel (CH)

(21) Appl. No.: 15/030,632

(22) PCT Filed: Oct. 21, 2013
(86) PCT No.: PCT/EP2013/071939

§ 371 Date: Apr. 20, 2016

(51) Int. Cl. A61B 5/0408; A61B 5/0492; A61N 1/04; A61B 5/053; A61B 5/00; A61B 5/0478; A61B 5/0496

(52) U.S. Cl. CPC A61B 5/04085; A61B 5/0478; A61B 5/0492; A61B 5/0496; A61B 5/053; A61B 5/6804; A61N 1/0452; A61N 1/0456; A61N 1/0484

(57) ABSTRACT

Body electrode system including a set of standalone electrodes units for measuring physiological signals of a body part and/or electrically stimulate a body part. A connecting garment provides electrical connection between each standalone unit of the set. Each unit of the set is individually positionable at a specific chosen position onto the body to be sensed and/or stimulated. The garment is electrically connectable to said units, preferably after placement of said set onto the body.

### FIELD

The present invention concerns a system and method to place, attach, and especially electrically connect sensing or stimulating standalone units measuring physiological signals or stimulating body parts.

### BACKGROUND

The most used method to measure biopotentials (e.g., ECG, EEG, EMG, EOG, etc.) is to place electrodes at specific body locations. Each electrode is electrically connected to a centralized electronic unit amplifying and acquiring the potential signals. The cables are insulated and preferably shielded. They connect the electrodes with the centralized electronic unit in a star arrangement, where the centralized electronic unit is the common point to which all cables converge. The electrode can be made of a piece of Ag/Ag+Cl— interfaced to the skin with a gel and affixed to the body skin with adhesive. Such electrodes are most of the time disposable and passive. Other types of electrodes include conductive material (metal, conductive rubber/silicone, conductive textile, conductive tape, etc.) in direct contact with the skin (dry electrodes), or interfaced to the skin with gel or liquid (water, saline, sweat, etc.). The electrodes may also physically perforate the skin to get a direct contact with the moisturized tissues under the dry upper layers of the skin.

The measurement of bioimpedance or the electrical stimulation of a body part is performed the same way as the measurement of biopotentials, except that current is additionally injected, at least at some electrodes.

The methods presented so far have the drawback to require many cables, resulting in a poor overall integration. Another method that somewhat alleviates this problem affixes on the body adhesive patches or bandages comprising two or more electrodes and sometime also including a miniaturized centralized electronic unit. In this case, the cables connecting the electrodes to the centralized electronic unit are no longer apparent, but embedded in the patch. However, the location of the electrodes in the patch is predetermined and cannot be randomly chosen by the practitioner.

Another similar method allowing concealing the cables is to embed them in a garment. The cables can even be made of conductive textile in some situations. In this approach, the electrodes are also part of the garment, which makes the donning and doffing as easy as to put on or take off a normal garment. As for the patches, this method results in a good overall integration but may suffer from the predetermined location of the electrodes or measurement points in case the practitioner wants to keep the control of the electrode locations. Moreover, a specific garment size and cut may be required to fit a given body size or shape.

Many products, systems and solutions are well known in the art, such as, for instance, Holter's ECG recorder. In this device, the adhesive electrodes are freely placed at any locations chosen by the practitioner. However, an insulated and shielded cable has to connect each electrode to the centralized electronic unit that amplifies and acquires the data. This results in a poor overall integration. In addition to the patient discomfort, such approach may suffer from accidental pulling of cable, which would make signal artifact if not totally interrupt the monitoring of the signals. All cables are connected to the centralized electronic unit in a star arrangement with the electronic unit being the convergence center.

Caridiolnsight provides a solution that allows measuring 250 ECG leads with electrodes embedded in a patch. In this product, the connection still follows the same topology as the one of the Holter's recorder. However, as it is a patch, the free choice of the electrode locations by the practitioner is lost.

Telzuit provides a patch integrating the electrodes, the cables, and the centralized electronic unit. The topology is still the same as the one of the Holter's recorder, namely a star arrangement with the center in the centralized acquisition electronics.

Weartech's smart shirt integrates textile electrodes. The electrical connections are made of conductive textile. They are not particularly shielded or insulated in this product, but this may degrade the signal quality especially in the presence of sweat or electromagnetic disturbances. The effect of these drawbacks is limited to an acceptable level in the targeted application by keeping the electrodes close to the centralized electronic unit.

Camtech's product consists of two electrodes. The location of one of them can be freely chosen by the practitioner. The other electrode should be placed at a distance from the first one determined by the cable length. The centralized electronic unit is located on top of one of the electrodes. The system is usually provided with two electrodes. However, if it were extended to more electrodes, the connection would still be a star arrangement with all cables converging to the centralized electronic unit. Moreover, the cable is conventional, i.e., insulated and shielded.

SenseCore also provides a known solution. The electrodes are standalone electrode-units or more generally sensing and stimulating standalone units. The product is made of only two units (one reference standalone unit and one measuring/injecting standalone unit). However, it could be extended to more measuring/injecting standalone units. In this case, they would be connected to the same electrical connection in any chosen arrangement, i.e., not limited to a star arrangement with its center at a specific point. Moreover, the electrical connection does no longer need to be shielded or insulated. In the SenseCore product, the standalone units are embedded in a shirt and placed on the body at predetermined locations: the practitioner cannot freely choose the location of the standalone units.

US2008091097 describes a garment preferably in the form of a body suit, which carries one or more sensors for sensing bodily functions of a wearer of the body suit. The body suit preferably has stretchable sections or belts upon which the sensors are carried such that the sensors are maintained in proper position on the body for reliable detection of the body functions.

U.S. Pat. No. 7,173,437 relates to a system for unobtrusively measuring bioelectric signals developed by an individual includes multiple sensors, one or more of which constitutes a capacitive sensor attached to a holding device. The holding device serves as a mounting structure that holds sensors in place within a wearable garment. The holding device and sensors are horizontally and vertically adjustable relative to the garment, while the sensors are pressed against the individual and prevented from undesirable shifting upon movement of the individual.

In US2003208830, a wearable garment having medical electrode/sensors mounted in predetermined position relative to the skin of a wearer provides for the sensors to be removed and attached easily for cleaning, repair and/or replacement without adverse effect on the electrical connections thereto. The garment comprises a wearable body structure having one or more flexible electrical conductors on its inner surface, with a first separable fastener half-section, made of flexible electrically conductive material, mounted on the inner surface and electrically connected to the electrical conductor. A medical sensor formed of flexible conductive material, preferably conductive silicone, is coupled, electrically and mechanically, to a second separable fastener half-section also formed of flexible conductive material. The second separable fastener half-section is configured to couple separably with the first half-section to complete an electrical current path between the medical sensor and the electrical conductor.

### SUMMARY

One aim of the present invention is to propose a system that is simpler and more compact than the known devices.

Another aim of the present invention is to propose a system that provides an enhanced ergonomics.

Still another aim of the present invention is to propose a system enabling removal and reinstallation of electrodes and that allows positioning the electrodes at selected locations onto the body.

According to the invention, these aims are achieved notably by means of the object of the main claim, whilst the dependent claims illustrate advantageous variant embodiments of the invention.

More particularly, the invention provides a body electrode system comprising a set of standalone electrodes units for measuring physiological signals of a body part and/or electrically stimulate a body part, a connecting garment providing electrical connection between each standalone unit of the set; each unit of the set being individually positionable at a specific chosen position onto the body to be sensed and/or stimulated; and the garment being electrically connectable to said units preferably after placement of said set onto the body.

Such system may be used with patients having different body shapes, instead of requiring a whole set of garment of different sizes.

The choice of standalone-unit types is independent of the choice of the garment size, thus, for different measurement configurations, the units and garment combination remains tractable in terms of equipment and do not required a large number of garments with embedded units to cover all combinations.

The standalone units may have to be placed by a practician. Placing standalone units that are hidden by a shirt/vest is difficult, especially if the relative placement of the units in the shirt/vest is approximately fixed. With the system of the invention, the standalone-unit placement is free.

In an embodiment, the garment also provides mechanical support for a set of units. The garment and standalone units are thus preferably provided with complementary releasable attachment members. The handling, cleaning and sterilizing of a garment without any unit is much easier.

Advantageously, the garment provides memory positioning of each unit with respect to each other, and/or with respect to its original specific position on the same body.

For instance, the standalone units may be secured with the garment (shirt/vest) after the first donning. The patient can then remove the garment, for example to take a shower, without taking the risk of moving the electrodes. Therefore, the electrodes will automatically be at the right locations when the patient will put on the garment again.

If the standalone units require single electrical connection, the garment is preferably provided with single insulated conductive layer, and said standalone units comprise a single connector extending from said unit.

If the standalone units require dual electrical connection, the conductive garment is preferably provided with dual insulated conductive layer, and said standalone units comprise a pair of connectors extending from said unit.

In a further variant, the set of units includes a reference electrode unit. Such reference is advantageous for instance if biopotentials are to be measured.

The complementary attachment members are preferably magnetic. In several variants, the complementary attachment members are provided with fasteners (Velcro, screws, clip, etc.). The units are thus easily removable, either for a new patient or a new measurement configuration or to provide easy handling, cleaning and sterilizing of a garment without any unit.

The conductive garment advantageously comprises a mesh and/or a shirt and/or a belt, and/or conductive tape to be pasted on the body and/or conductive wire or yarn.

The invention also provides a method to install onto a body and electrically connect a body electrode system as previously described, comprising the following steps:

Affix individually at least two units of the set of standalone electrode units on the body to be sensed and/or stimulated at specific chosen locations of the body (for instance with adhesive, suction cup, bandage, pressure, weight, etc.); and

electrically connect the garment of the body electrode system to all (previously) affixed standalone units (and reference electrode if any).

The method can further comprises attaching each unit of the set to the garment using complementary attachment members provided on said standalone units and said garment.

If biopotentials are to be measured, the method can further comprises selecting a reference electrode (passive or active electrode, or preferably a reference standalone electrode-unit) and affixing it on the body at a specific chosen location.

The invention proposes a method that at the same time benefits from a free placement of the electrodes and from the absence of explicit cables. This provides a very good integration, increases the subject comfort, and reduces the risk of inadvertently pulling a cable.

### BRIEF DESCRIPTION OF THE FIGURES

Examples of embodiments of the invention are illustrated by the attached figures wherein showing electrical connection between garment and standalone units;

FIGS. 1A to 1D illustrate schematic examples of body electrode systems of to the invention, showing electrical connection between garment and standalone units;

FIG. 2 illustrates schematic examples of body electrode systems of to the invention, showing mechanical connection (magnetic variant) between garment and standalone units;

FIG. 3 illustrates schematic examples of body electrode systems of to the invention, showing mechanical connection (spring member variant) between garment and standalone units;

FIGS. 4 and 5 illustrate schematic examples of body electrode systems of the invention, showing detailed examples of electrical connection with one or two conductive links.

### EXAMPLE(S) OF EMBODIMENTS

In this document, a “specific chosen position” corresponds to: a position on a body as determined by a user (such as a practitioner). Depending on the measures to be made and/or the stimulations to be performed, the user determines, based on his knowledge and/or installation rules and practice, the best position for each unit of the set on the body. The user may optionally decide to use a specific type of sensor or electrode in relation to the body part characteristics and/or the measurement types or stimulations to be provided. Optionally, before installation, the user provides an electrode mapping, providing details of electrodes to be installed and visual references or landmarks to allow easy placement on the body.

In this document, “initial specific chosen position” means: the position of a unit on a body part as determined by a user when the units where initially placed on the body by said user, before any removal of the entire set, and preferably before electrical connection of the set.

In this document, “standalone electrodes units” means: electrode devices that are configured and designed to be initially placed on a body, such as a human body, preferably without being linked or connected to any other electrode or piece of garment or electrical connector or wiring, although electrical connection may be required between a set of standalone units for various cases as mentioned below. This enables easy and quick placement of a set of electrodes without interference by a non-electrode component. The invention relies on standalone units, i.e., each of them having its own battery and sensor and/or stimulation electronics as well as all electronics to manage all required functions and to communicate with other devices). Therefore, other signals than potentials and impedances can be simultaneously measured, for instance, photo-plethysmography, pulse oximetry, sounds, skin temperature, core body temperature, etc. More stimulation types are also possible, such as acoustical (vibration), optical, magnetic, thermal, pressure, etc. being for its direct effect on the body or with the goal to measure the body reaction to the stimulation. Ambient and contextual signals can also be captured, such as acceleration, angular acceleration (gyro), magnetic field, temperature (air, water), humidity, pressure (air, water), GPS, radiation, pollutant, chemicals, pH, etc. Finally, an explicit centralized electronic unit is no longer necessary.

A standalone unit comprises on its external housing a conductive part, not in electrical contact with the skin, that can be electrically connected with other standalone units.

In this document, “memory positioning” relates to: a set of standalone electrodes that are placed on a body at specific chosen positions, and mechanically connected between each other such that if the set is removed from the body, for instance between two or more series of measurements, the set is easily and quickly repositionable on the same body (or same body part) with each standalone unit of the set recovering its previous respective position and/or its previous location on the body.

Electrical connection between a set of standalone units may be required for various cases, such as, for example:
the reference potential of biopotential measurements (ECG, EEG, EOG, EMG, etc.); and/or
the current return resulting from the current injected through the skin under the standalone unit for bioimpedance measurements (for respiration, EIT, hydration, visceral fat, etc.); and/or
the current return resulting from the current injected through the skin under the standalone unit for electrical stimulation (muscles, nerves, brain, etc.); and/or
the broadcast of synchronization information by one standalone unit to all others; and/or
the data concentration by one standalone unit collecting the data sent by all others; and/or
the recharging of the standalone units.

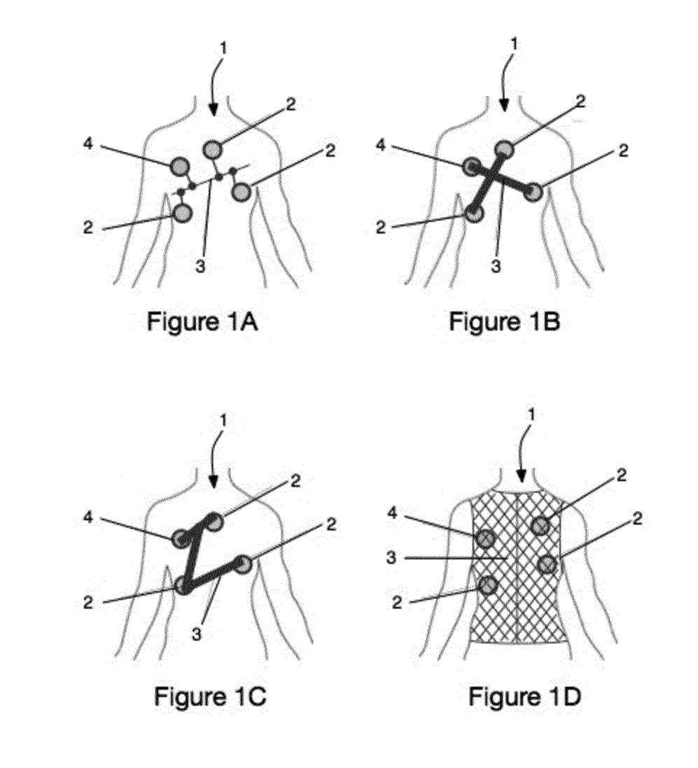

FIGS. 1A to 1D illustrate schematic examples of body electrode systems 1 according to the invention. FIG. 1A illustrates details of the system connecting topology, with a set of standalone electrode units 2 that are electrically linked using a garment 3. A reference standalone unit 4 may also be added to the set. FIGS. 1B and 1C shows examples of embodiments in which the units 2 are connected by a garment 3 taking the form of a conductive tape or ribbon. In these examples, the tape also serves as an attachment means to affix the units 2 to the body.

FIG. 1D illustrates a preferred embodiment in which connection is provided with a garment 3 taking the form of a conductive mesh or garment worn on top of the affixed standalone units. This embodiment is particularly advantageous because, as explained in more details hereafter, it enables the wearer of the garment to remove the latter, together with the electrode units: when required, the garment may be reinstalled, with all electrode units 2 recovering their initial respective positions.

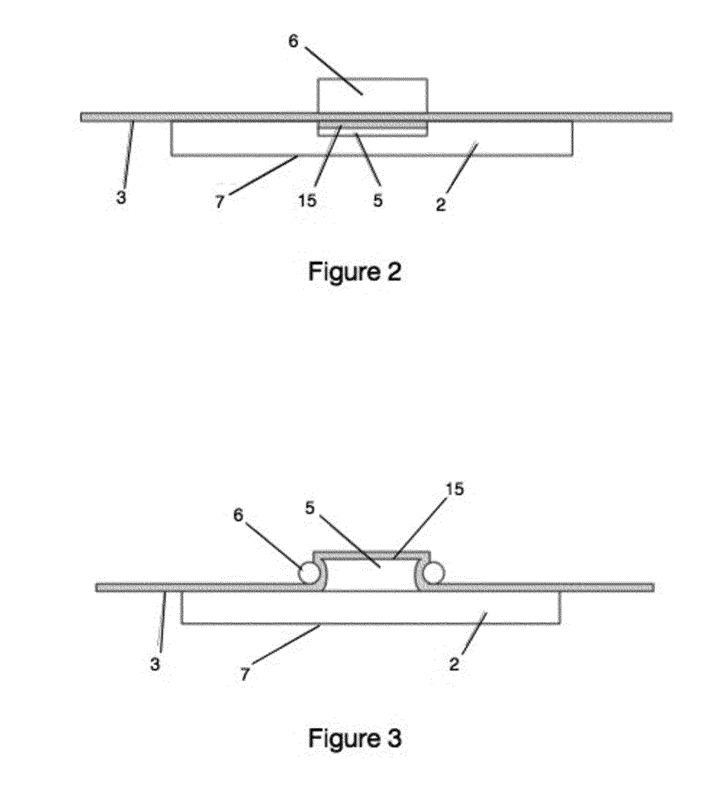

The embodiments illustrated in FIGS. 2 and 3 show various examples of complementary releasable attachment members, with member 5 on the standalone unit 2 and member 6 on the garment 3. FIG. 2 illustrates an embodiment in which the garment 3 is fastened with the standalone units 2 with a magnet attracted by a magnetic material or the opposite. A conductive portion 15 on the top side of the standalone unit is compressing the conductive fabric 3 to provide a good contact and conductivity. In the embodiment illustrated in FIG. 3, the garment 3 is fastened with the standalone units 2 with a spring, elastic, or the like compressing the conductive fabric on the conductive portion 15 of the standalone unit.

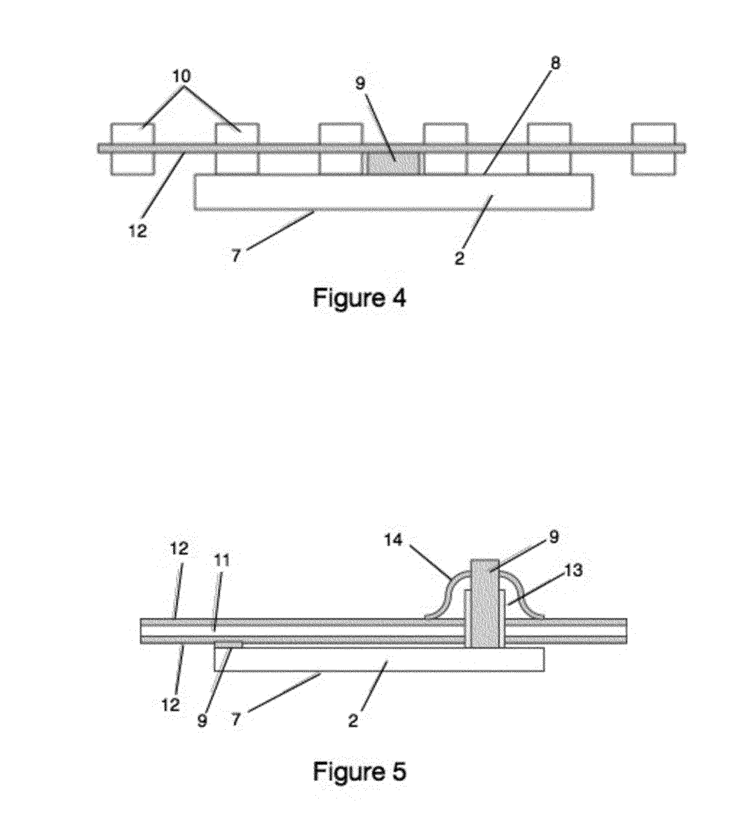

FIG. 4 illustrates insulated spacers 10, as part of conductive link 12 made with conductive garment 3, tape, or yarn/wire, that connects with standalone units 2 via a connector 9 protruding from the standalone units. Such spacers 10 prevent any possibly unwanted direct electrical contact with the conductive link 12, in particular with the body.

FIG. 5 illustrates an arrangement having a dual conductive layer 12 and a dual connector 9 configuration. The two conductive layers 12 are separated by an insulation layer 11. The standalone units 2 are affixed on the skin, with one face 20 in interaction with the skin and the other side comprising a first connector 9 connecting with a first conductive layer 12 and a second connector 9 protruding so as to perforate the garment or tape and connecting with a second conductive link 12 of garment or tape via a conductive fastener 14. The second connector 9 is insulated in its base with an insulation tube 13 so as not to connect with the first link of the garment or tape.

The invention also relates to a method for installing onto a body and electrically connecting a body electrode system as previously described. In this method, at least two standalone units 2 are used for measuring physiological signals and/or stimulating body parts and/or measuring ambient and contextual signals. The standalone units are affixed individually on the body (e.g., by the practitioner), in order to be sure they are in the right place before connecting them. A connecting garment 3 embodied as a shirt or tissue or mesh or tape or yarn/wire is used to connect all units 2 of the set. The electrical connection enables setting a common potential to all standalone units, allows injected-current return, allows synchronization and data concentration, possibly allows recharging the standalone units. In a preferred variant, the standalone units 2 are mechanically secured with the garment 3. Thus, the garment can be easily removed and put on again with the standalone units placed at the same locations.

Note that the connection does not have to be a star arrangement or to converge to a centralized electronic unit or to a particular standalone unit. As a matter of fact, there is no need to have any centralized electronic unit since every unit is standalone and plays the role of a fraction of the centralized electronic unit. Any connecting arrangement is allowed as long as there is an electrical connection with all units 2 and (if any) the reference electrode 4.

The standalone units 2 can therefore acquire the signals or stimulate the body parts from where they are placed. The measurement of potentials is performed relative to the reference potential of the electrical connection linking the standalone units. All standalone units can operate in concert by exchanging some synchronization information, for instance with wireless transmission, or using the electrical connection that links them, as disclosed for instance in EP2,567,657A1, included herewith by reference.

The reference electrode 4 should preferably connect to the inside of the body with low impedance. Normal dry-skin impedance may be of relative high impedance, typically over several 100 kΩ cm2. Low impedance can be obtained with large contact surface. For instance for 1000 cm2, the impedance is about 100Ω. With wetted or gel electrodes, the impedance is about ten times lower and with active electrodes or preferably with dry standalone electrode-units—such as disclosed in US20110001497—typically over 1000 times lower for the same contact area. Low impedance reduces mains disturbances and avoids the need of shielded or insulated connections for biopotential measurements. For bioimpedances, it reduces the effect of skin impedance variations (due to motion, moisture, pressure, etc.).

If biopotentials are measured, the electrical connection (garment, tape, wire, etc.) of the standalone units 2 can play the role of passive reference electrode if it is explicitly in contact with the skin. In all other cases, the connection can either be in electrical contact with the skin or insulated from the skin. If, for any reason, the latter is preferred, the insulated spacers 10, e.g., beads, can be used to keep the electrical connection away from direct contact with the body skin, as illustrated in FIG. 4.

On the other hand, the connector 9 of the standalone units must be shaped in such a way that it can reach the conductive link 12 between the insulated spacers 10.

For standalone units requiring a double connection (two-wire bus), the method can be extended to garments (or tapes) comprising an arrangement of two conductive links 12 separated by a layer of insulating fabric 11, or, in a variant, a fabric that has been made conductive on both sides, for instance by plasma deposition of a metal (e.g., Ag). A possible arrangement of standalone units with such garment (or tape) is shown in FIG. 5. The fastener 14 can be a spring applying its force between the connector 9 and the remaining part of the standalone unit so as to compress the two conductive links 12 and thus ensure a good electrical contact with the conductive layer 12 of the garment/tape. The connector 9 can also take advantage of this compression by being placed below the fastener so as to improve its contact with the conductive layer 12. The fastener 14 can also be, for instance, a screw, or a magnet attracted by a magnetic material being part of the standalone unit.

In such a case, the method is adapted as following:

taking at least two standalone units and affix them on the body at chosen locations (for instance with adhesive, suction cup, bandage, pressure, weight, etc.);

optionally but preferably if biopotentials are measured, taking a reference electrode (passive or active electrode, or preferably a reference standalone electrode-unit) and affix it on the body at a chosen location;

connecting together all standalone units and reference electrode (if any) by putting on a garment or tape comprising two conductive layers separated by an insulated layer;

using fasteners to connect the outer conductive layer of the garment or tape with the protruding part of the standalone units.

Note that in a further variant, instead of using standalone units with the protruding part described above, the fastener is used to perforate the garment or tape, like a screw or a pin/nail. This has the advantage of allowing the garment to freely slide on the standalone units before its fastening.

The advantage of the two-wire bus allows being less sensitive to impedance variation of a single connection (for instance due to changing tension on parts of the conductive textile), and can simplify the communication between the standalone units. In addition, the two-wire bus can be used to recharge the standalone units when not worn.

The standalone units fastened with the garment may also be an advantage by allowing the subject to temporarily remove the garment together with the standalone units and then automatically find the standalone units at the same locations by putting on the garment again. This of course works also for a single electrical connection with the garment. In this case the fastener may or may not be used as electrical connection and does not have to perforate the garment (see FIG. 5).

Compared with the state of the art where the electrode-units are fasten before the first donning at predetermined locations, this approach allows the practitioner to freely choose the initial locations that will be ‘memorized’ by the garment for subsequent donnings.

Other possibility of fastener for this variant includes the one of FIG. 2. Still other variants of fastener could be obtained with conductive velcro or adhesive. All these types of fastener can be used also with straps (non-adhesive tape) which have the advantage compared to an adhesive tape not to stick to hair or skin.

### LIST OF REFERENCE NUMBERS

1 Electrode system
2 Standalone electrode units
3 Connecting garment
4 Reference standalone unit
5 Complementary releasable attachment members on the standalone unit 2
6 Complementary releasable attachment members on the garment 3
7 Skin face of electrode
8 Connecting face of electrode
9 Connectors
10 Insulation spacers
11 Insulation layer
12 Conductive links
13 Insulation cover
14 Conductive fastener
15 Conductive portion

What is claimed is:

1. Body electrode system comprising a set of standalone electrodes units for measuring physiological signals of a body part and/or electrically stimulate a body part, a connecting garment providing electrical connection between each standalone unit of the set,
each unit of the set being individually positionable at a specific chosen position onto the body to be sensed and/or stimulated;
each unit comprising a face arranged to interact with the skin of the body when the unit is positioned onto the body; and
each unit comprising a conductive part electrically connectable to the garment to electrically connect said units of the set to the garment wherein the conductive part is not in electrical contact with the face;
such that the garment is electrically connectable to said units independently of the chosen position onto the body of each of the units.

2. Body electrode system according to claim 1, wherein the garment also provides mechanical support for the set of units.

3. Body electrode system according to claim 1,
wherein the garment provides memory positioning of each unit with respect to each other.

4. Body electrode system according to claim 1,
wherein the garment provides memory positioning of each unit with respect to its original specific position on the same body.

5. Body electrode system according to claim 2,
wherein said garment and said standalone units are provided with complementary releasable attachment members.

6. Body electrode system according to claim 1,
wherein, when standalone units require single electrical connection, garment is provided with single conductive link, and said standalone units comprise a single connector extending from said unit.

7. Body electrode system according to claim 1,
wherein when standalone units require dual electrical connection, connecting garment is provided with dual conductive link insulated one from the other by insulated layer, and said standalone units comprise a pair of connectors extending from said unit.

8. Body electrode system according to claim 1,
wherein the set of units include a reference electrode unit.

9. Body electrode system according to claim 1,
wherein the complementary attachment members are magnetic.

10. Body electrode system according to claim 1,
wherein the complementary attachment members are provided with fasteners.

11. Body electrode system according to claim 1,
wherein the conductive garment comprises any one of: a conductive wire, a yarn, a belt, a mesh or a shirt.

12. Body electrode system according to claim 1,
wherein the conductive garment comprises a conductive tape.

13. A method to install onto a body and electrically connect a body electrode system comprising a set of standalone electrodes units for measuring physiological signals of a body part and/or electrically stimulate a body part, a connecting garment providing electrical connection between each standalone unit of the set, each unit of the set being individually positionable at a specific chosen position onto the body to be sensed and/or stimulated; each unit comprising a face arranged to interact with the skin of the body when the unit is positioned onto the body; and each unit comprising a conductive part electrically connectable to the garment to electrically connect said units (2) of the set to the garment; wherein the conductive part (9, 15) is not in electrical contact with the face;
the method comprising the steps of:
affix individually at least two units of the set of standalone electrode units on the body to be sensed and/or stimulated at specific chosen locations of the body; and
electrically connect the garment of the body electrode system to all affixed standalone units;
wherein the garment is electrically connected to said units independently of the chosen position onto the body of each of the units.

14. The method of claim 13,
further comprising attaching each unit of the set to the garment using complementary attachment members provided on said standalone units and said garment.

15. The method of claim 13,
further comprising, if biopotentials are to be measured, selecting a reference electrode and affixing it on the body at a specific chosen location.
